(19) United States
(12) Patent Application Publication
(10) Pub. No.: US 2021/0113750 A1
(43) Pub. Date: Apr. 22, 2021

(54) BABY/INFANT/TODDLER NASAL ASPIRATOR

(71) Applicant: TnC-AnS Solutions, LLC, Bozeman, MT (US)

(72) Inventors: Todd A. Roemmich, Simpsonville, SC (US); Carl S. Warren, Athens, GA (US); Stephen McAlister Jones, JR., Greer, SC (US); Steve Birrell, Bozeman, MT (US)

(21) Appl. No.: 17/073,521

(22) Filed: Oct. 19, 2020

Related U.S. Application Data
(60) Provisional application No. 62/924,224, filed on Oct. 22, 2019.

(51) Int. Cl. A61M 1/00

(52) U.S. Cl. CPC A61M 1/0041; A61M 1/008; A61M 2240/00; A61M 2210/0618; A61M 2205/073; A61M 1/007

(57) ABSTRACT

Provided is a manually operated nasal aspirator having an elongated cylinder body and a piston. The cylinder body has a sidewall, a nozzle at a forward end, and an elongated slot extending longitudinally in the sidewall to a rearward end. The piston is configured to slide within the cylinder body between first and second positions and has an actuator laterally extending therefrom through the elongated slot. The piston is manually movable between the first and second positions by a user and is not biased by any other means so that it will rest at all positions between the first and second positions when not being manually manipulated. The cylinder has a length sufficient to fully contain the piston therein in both the first and second positions.

### RELATED APPLICATIONS

This application claims priority to U.S. Provisional Patent Application No. 62/924,224, filed Oct. 22, 2019, and incorporates the same herein by reference.

### TECHNICAL FIELD

This invention relates to aspirators that are manually operated (i.e., by hand and not mechanically driven or orally suctioned) to remove mucus from a newborn, infant, or toddler's nose.

### BACKGROUND

The noses of newborns are suctioned shortly after birth to enhance their breathing. In addition, infant and toddler nasal cavities can become clogged and normal breathing can be inhibited by a variety of factors, including mucus secreted in response to viruses and bacteria. Thus, newborns, infants, and toddlers often need their noses cleared of mucus.

There are many aspiration devices which address the general problem of clearing fluids, debris, mucus, and secretions from the nasal passages. The most common are manually operated aspirators that include a bulb that is squeezed to create a partial vacuum that sucks nasal discharge from the child's nostril (e.g., U.S. Pat. No. 6,290,667). These bulb aspirators normally include an oval shaped bulb with a tip and/or a stem. The bulb is generally made of a rubber or an elastomeric type of material that has an opening into an inner hollow cavity.

A bulb aspirator is used manually by squeezing (compressing) the bulb forcing air out of the inner hollow cavity through an opening in the tip. The tip is then placed in the nasal passage of the child to create a seal between the tip and the nasal passage. Once in place, the compressed bulb is released sucking nasal fluid (mucus, etc.) into the inner hollow cavity of the bulb.

Bulb aspirators suffer from the following disadvantages:
a. It is difficult to evenly control the degree of suction created by compressing the bulb;
b. It is difficult to thoroughly clean the inner cavity of the bulb;
c. The amount of mucus removed cannot be accurately monitored or determined; and/or
d. For many operators, it is unnatural or counterintuitive to squeeze the bulb “before” inserting the tip into the child's nostril. As a result, an operator may place the tip into the child's nostril and then squeeze the bulb, which blows air into the nostril and hinders the removal of mucus.

Other manually operated aspirators use a mouthpiece that the operator uses to suck fluid (mucus, etc.) from the child's nostril. These mouthpiece aspirators typically include of a collection cylinder with a tip for collecting nasal discharge, a tube with a mouthpiece at one end and a cap at the other end cap for attaching to the collection cylinder, and a filter made of a foam-type of material.

The mouthpiece aspirator is used manually by placing the filter in the collection cylinder and attaching the cap of the mouthpiece tube to the collection cylinder. See, for example, U.S. Pat. No. D678,501. The tip of the collection cylinder is then placed in the nasal passage of the child to create a seal between the tip and the nasal passage. Once in place, the operator sucks into the tube using the mouthpiece which sucks (draws by vacuum) nasal fluid (mucus, etc.) into the collection cylinder.

Mouthpiece aspirators suffer from the following disadvantages:
a. It is psychologically offensive for many (parents, nurses, etc.) to suck snot (mucus, etc.) from a child's nose;
b. Filters must be replaced frequently, which increases the cost of operating and maintaining the aspirator;
c. The initial purchase cost is more than bulb aspirators; and/or
d. The amount of mucus removed cannot be accurately monitored or determined.

Less commonly used are mechanical aspirators that are battery-operated or spring-loaded. These aspirators come in variety of designs. See, for example, U.S. Pat. No. 6,517,511. In these aspirators, a partial vacuum for suctioning the nose is created mechanically by a battery or spring-operated pump.

Battery and spring-loaded aspirators suffer from the following disadvantages:
a. The initial purchase cost is more than bulb or mouthpiece aspirators;
b. The power source (battery or spring) for creating partial vacuum must be replaced over time thus, increasing the cost of maintaining the aspirator; and/or
c. The battery or spring may unexpectedly fail, thus preventing the user from suctioning the child's nose on a timely basis.

There is significant need for an aspirator that achieves the following:
a. is easy to operate manually;
b. is not psychologically offensive to use;
c. is operated by one hand while holding a newborn or infant;
d. controls the degree of suction;
e. shows the amount of mucus (discharge) irrigated;
f. is easily disassembled for cleaning;
g. is easily re-assembled for use;
h. is not subject to surprise mechanical failure.

### SUMMARY

This invention is a manually operated device for the aspiration and removal of fluids from the body. Specifically, this invention is an aspirator for removing mucus, debris, and secretions from the nose. The invention is primarily designed for newborns, infants, and toddlers.

The present invention provides a manually operated nasal aspirator having an elongated cylinder body and a piston. The cylinder body has a sidewall, a nozzle at a forward end, and an elongated slot extending longitudinally in the sidewall to a rearward end. The piston is configured to slide within the cylinder body between first and second positions and has an actuator laterally extending therefrom through the elongated slot. The piston is manually movable between the first and second positions by a user and is not biased by any other means so that it will rest at all positions between the first and second positions when not being manually manipulated. The cylinder has a length sufficient to fully contain the piston therein in both the first and second positions.

This invention overcomes shortcomings and disadvantages of prior art and suctions nasal passages in a simple, cost-effective manner. The invention is easily operated with one hand while holding a newborn or infant. The invention allows the user to control the amount of suction and monitor the amount of mucus (discharge) removed. The invention creates suction (vacuum) manually and thus, is not subject to surprise, mechanical failure. The invention is not psychologically offensive to use.

Other aspects, features, benefits, and advantages of the present invention will become apparent to a person of skill in the art from the detailed description of various embodiments with reference to the accompanying drawing figures, all of which comprise part of the disclosure.

### BRIEF DESCRIPTION OF THE DRAWINGS

Like reference numerals are used to indicate like parts throughout the various drawing figures, wherein:

FIG. 1 is an isometric view of a nasal aspirator according to one embodiment of the present invention;

FIG. 2A is a cross-sectional view thereof with an internal piston in a first position;

FIG. 2B is a similar view with the piston in a second position; and

FIG. 3 is an enlarged cross-sectional view thereof.

### DETAILED DESCRIPTION

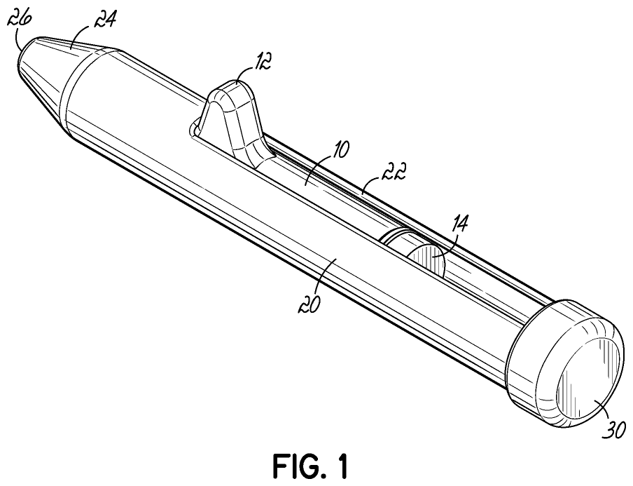

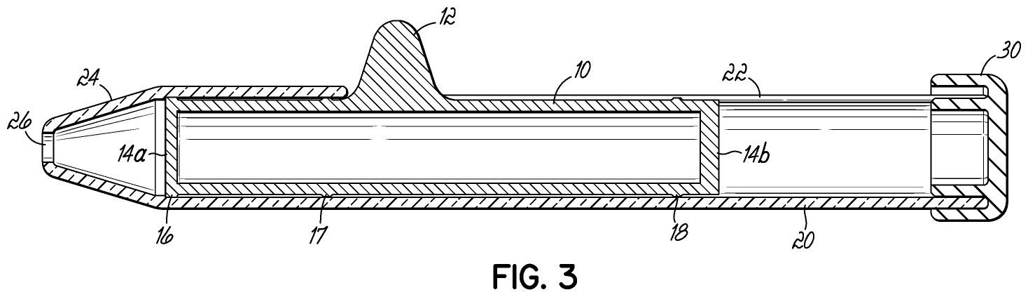

This invention is a multi-purpose newborn, infant, toddler nasal aspirator. As shown in FIGS. 1 and 3, one embodiment of the invention has three main components: an inner piston 10 with a thumb-rest 12 and at least one end wall 14a, 14b, an outer cylinder 20, which may be made of clear material, such as plastic; and an end cap 30.

The three components can be made of, for example, polypropylene, which is compliant with Federal Drug Administration (FDA) standards for biocompatibility. The thumb rest 12 can extend through a longitudinal slot 22 in the wall of the outer cylinder 20 to allow control of the piston 10. The outer cylinder 20 can have a conical nozzle 24 with a central opening 26 that is smaller in cross-section that the piston 10. This increases the flow thought the opening 26 as the piston 10 is withdrawn to create an internal vacuum. Changeable nozzles (not shown) may be used to adjust size, but the illustrated embodiment provides simplicity. Unlike a typical syringe whose plunger is operated from the end, the manner in which this device is gripped, with the thumb moving the thumb rest 12 protruding through the side wall of the cylinder 20, provides a more natural grip and controlled movement.

The invention is assembled by sliding the inner piston 10 into the outer cylinder 20. Referring now in particular to FIG. 3, the inner piston 10 can have one or more “O-ring” type annular protrusions 16, 17, 18 that can create and seal at least a partial vacuum. A first annular protrusion 16 is located at or adjacent to one end of the inner piston 10 nearest the nozzle 24. A second annular protrusion 17 may be located at or adjacent the thumb rest 12 and a third annular protrusion 17 may be located at or adjacent the end of the piston 10 opposite the first annular protrusion 16. In addition to creating a vacuum (primarily by the first annular protrusion 16), these three “O-ring” type protrusions 16, 17, 18 also stabilize movement of the inner piston 10 within the outer cylinder 20 by providing bearing surfaces. The end cap 30 is then attached to retain the piston 10, if desired.

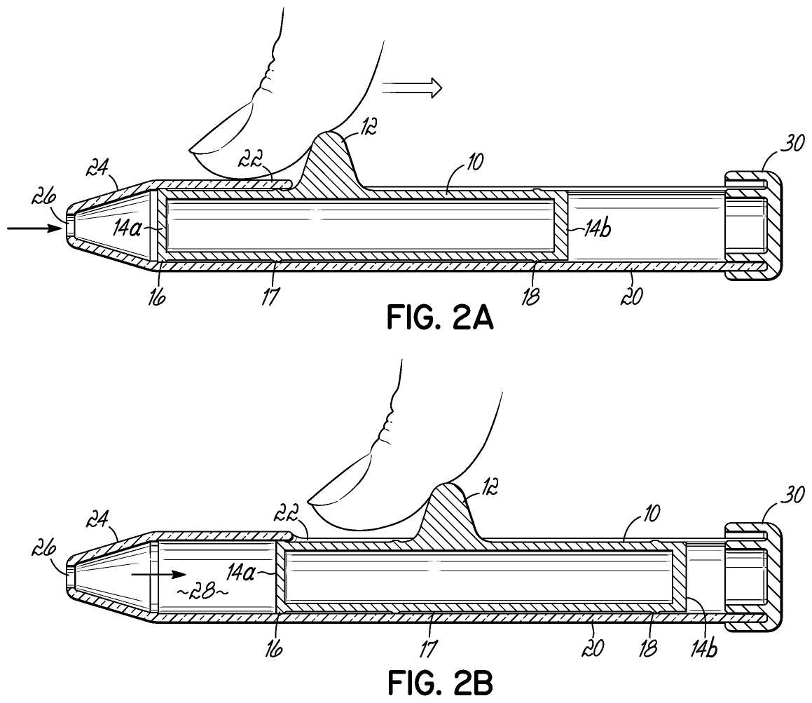

With reference to FIGS. 2A and 2B, the invention is used by placing the tip 24 in the nasal passage of the child to create a seal between the tip 24 and the nasal passage. The operator then uses the thumb-rest 12 on the inner piston 10 to slide the inner piston 10 away from the child (toward the operator or rear end of the cylinder 20) and, in doing so, suctions the child's mucus. The mucus is collected in the space 28 created within the outer cylinder 20 and forward end wall 14a of the inner piston 10 as the inner piston 10 is slid away from the nozzle 24. If the outer cylinder 20 is clear plastic, the operator can monitor the amount of mucus collected. If the aspirator is filled with collected mucus, the operator can discharge the collected mucus by sliding the inner piston 10 toward the nozzle 24 of the aspirator. The operator can then collect additional mucus by placing the nozzle 24 into the child's nasal passage and repeating the process.

Because the slot 22 extends to the rear end of the cylinder 20, the device can be easily cleaned by removing the end cap 30 and sliding the inner piston 10 out of the outer cylinder 20. Once disassembled, all three components can be thoroughly cleaned and easily reassembled.

While one or more embodiments of the present invention have been described in detail, it should be apparent that modifications and variations thereto are possible, all of which fall within the true spirit and scope of the invention. Therefore, the foregoing is intended only to be illustrative of the principles of the invention. Further, since numerous modifications and changes will readily occur to those skilled in the art, it is not intended to limit the invention to the exact construction and operation shown and described. Accordingly, all suitable modifications and equivalents may be included and considered to fall within the scope of the invention, defined by the claim or claims.

What is claimed is:

1. A manually operated nasal aspirator, comprising:
an elongated cylinder body having a sidewall and a nozzle at a forward end, an elongated slot extending longitudinally in the sidewall to a rearward end;
a piston configured to slide within the cylinder body between first and second positions and having an actuator laterally extending therefrom through the elongated slot, the piston being manually movable between the first and second positions by a user and not biased by any other means so that it will rest at all positions between the first and second positions when not being manually manipulated; and
the cylinder having a length sufficient to fully contain the piston therein in both the first and second positions.

2. The nasal aspirator of claim 1, wherein the piston includes an annular seal bearing adjacent the forward end of the piston and in contact with an inner surface of the cylinder body.

3. The nasal aspirator of claim 3, wherein the piston includes at least one annular guide bearing in contact with the inner surface of the cylinder body.

4. The nasal aspirator of claim 1, wherein the cylinder body has an open rearward end and further comprising a removeable cylinder body end cap configured to close the open end and retain the piston within the cylinder body.
